(19) United States
(12) Patent Application Publication
(10) Pub. No.: US 2022/0331506 A1
(43) Pub. Date: Oct. 20, 2022

(54) FLUID REMOVAL DEVICE

(71) Applicant: KNOCKIN' BOOTS LLC, San Marcos, CA (US)

(72) Inventor: Brittany Amber Rafuson, San Marcos, CA (US)

(21) Appl. No.: 17/698,074

(22) Filed: Mar. 18, 2022

Related U.S. Application Data
(60) Provisional application No. 63/175,845, filed on Apr. 16, 2021.

(51) Int. Cl. A61M 1/00

(52) U.S. Cl. CPC A61M 1/682; A61M 2210/1475

(57) ABSTRACT

A body cavity fluid removal device includes a shaft having a first end, a second end opposite the first end, and an opening at the first end. A handle has a compressible body defining an interior volume. The handle is removably coupleable to the second end of the shaft. The handle is configured for inducing a suction force at the opening of the shaft.

### FIELD OF DISCLOSURE

The present disclosure is directed to a fluid removal device, and in particular, a fluid removal device for personal hygiene.

### BACKGROUND

Historically, people have used douches to wash the vagina, remove semen to prevent pregnancy, and reduce contracting a sexual transmitted infection. Vaginal douching includes filling a bottle with water and/or other ingredients, such as baking soda, iodine, antiseptics, or fragrance, and delivering the bottle's contents upward and into the vagina. Not only does douching fail to achieve the above-mentioned purposes (i.e., avoid pregnancy or reduce transmittal of STIs), but douching can disrupt the natural microbiome of the vagina, remove healthy bacteria, thereby allowing bad bacterial to multiply, and increase risk of infections, irritations, and other complications. While the American College of Obstetrics and Gynecology states that people should not use vaginal douches, vaginal douches are still being marketed and sold as effective vaginal hygiene products.

### SUMMARY

A fluid removal device of the present disclosure simply and safely removes fluid from a body cavity without flushing the body cavity with fluid. In one example application, the fluid removal device may remove semen from a vaginal canal after sex by applying a light and gentle suction to the device to collect the semen in a reservoir. The device is easily disassembled for cleaning and is reusable.

In accordance with a first aspect of the present disclosure, a body cavity fluid removal device may include a shaft having a first end, a second end opposite the first end, and an opening at the first end. A handle may include a compressible body defining an interior volume. The handle may be removably coupleable to the second end of the shaft. The handle may be configured for inducing a suction force at the opening of the shaft.

In accordance with a second aspect of the present disclosure, an aspirator for removing fluid from a body cavity may include a shaft having a first end, a second end, and a body having a curved surface extending between the first and second ends. The body may define a cavity. A handle may be coupled to the second end of the shaft and may include an interior volume in fluid communication with the cavity of the shaft.

In accordance with a third aspect, a method of removing fluid from a body cavity may include inserting a shaft of a device into a body cavity. The shaft may include a first distal end defining an opening and a second end coupled to a handle. The shaft may include a bore. The method may include activating a suction force through the shaft.

In accordance with the first, second, and third aspects, a body cavity fluid removal device, an aspirator for removing fluid from a body cavity, and/or a method of removing fluid from a body cavity may include any one or more of the following forms.

In one form when the handle is coupled to the shaft, the interior volume may be in fluid communication with the opening of the shaft.

In another form, the shaft and pump may couple together by a rotatable locking mechanism.

In one example, when the pump and shaft are securely coupled, an adapter of the handle may be disposed in a bore defined by the second end of the shaft.

In another example, the shaft may be curved.

In some forms, the shaft may include a wall having a variable thickness between the first end and the second end.

In other forms, a hole may be disposed through a wall of the handle.

In one example, the hole may be in fluid communication with a cavity of the shaft.

In some examples, a first hole may be disposed through a wall of the pump.

In another example, a second hole may be disposed through a wall of the shaft.

In these or other examples, when the shaft and the pump are securely coupled, the first and second holes may align to form a continuous vent in fluid communication with an interior cavity of the shaft.

In an alternative form, the second end may define a mating surface.

In one example, the pump may define a corresponding mating surface.

In these or other examples, the mating surfaces of the second end and the pump may seal in engagement when the shaft and pump are coupled.

In one form, the curved surface may include a point of inflection at the first end of the shaft.

In another form, shaft may be a medical grade silicone.

In one example, when the handle is compressed and released, a suction force may be created in the cavity of the shaft.

In another example, the body may be tapered and configured for insertion into a vaginal canal.

In some examples, the first end may define an opening having a diameter in a range of approximately 0.2 inches to 0.3 inches.

In these or other forms, a hole may be disposed through the wall of the handle.

In one example, the hole may be in fluid communication with the interior volume.

In other examples, a coupling mechanism may be arranged for removably coupling the handle to the shaft.

In some forms, activating a suction force may include compressing a flexible body of the handle externally disposed relative to the body cavity.

In one example, the handle may include a body defining an interior volume in fluid communication with the bore of the shaft.

In other forms, the method may include releasing the handle, causing fluid disposed in the body cavity to enter the shaft through the opening and be received by the interior volume of the handle.

### BRIEF DESCRIPTION OF THE DRAWINGS

FIG. 1 is a perspective view of a fluid removal device assembled in accordance with the teachings of the present disclosure;

FIG. 2 is an exploded, perspective view of the device of FIG. 1;

FIG. 3 is a partial, representative illustration of using the device of FIG. 1 to retrieve fluid in the vagina;

FIG. 4 is a top view of the device of FIG. 1;

FIG. 5 is a side view of the device of FIG. 1;

FIG. 6 is a side cross-sectional view of the device of FIG. 1;

FIG. 7 is a cross-sectional view of the device taken at 7-7 of FIG. 1;

FIG. 8 is a different cross-sectional view of the device taken at 8-8 of FIG. 1;

FIG. 9 is a perspective view of a handle of the device of FIG. 1;

FIG. 10 is a cross-sectional front view of the handle of FIG. 9;

FIG. 11 is a top view of the handle of FIG. 9;

FIG. 12 is a side, perspective view of a shaft of the device of FIG. 1;

FIG. 13 is a back, perspective view of the shaft of FIG. 12;

FIG. 14 is a top view of the shaft of FIG. 12;

FIG. 15 is a side view of the shaft of FIG. 12;

FIG. 16 is a side, cross-sectional view of the shaft of FIG. 12;

FIG. 17 is a partial view of the device of FIG. 1, showing the handled coupled to the shaft in a first coupling arrangement;

FIG. 18 is a different coupling arrangement between a pump and a shaft assembled in accordance with the teachings of the present disclosure; and

FIG. 19 is a partial view of a device like the device of FIG. 1, showing a different example vent hole.

### DETAILED DESCRIPTION

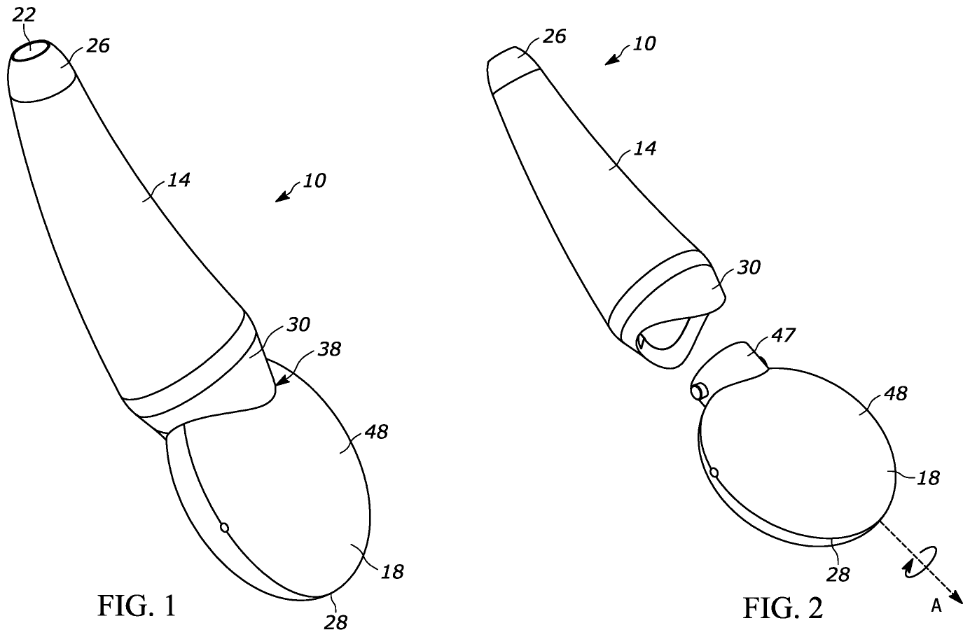

Generally speaking, a personal hygiene device 10 for removing a fluid from a body cavity is provided in FIGS. 1-5. The device 10 (also referred herein as an “aspirator” and a “fluid removal device”) includes a shaft 14 shaped and sized for insertion into a body cavity, such as a vagina or anal cavity, and a handle 18 removably coupled to the shaft 14. As shown in FIG. 2, the aspirator 10 can be disassembled for cleaning by twisting the handle 18 about an axis A and pulling the handle 18 away from the shaft 14. After cleaning both the handle 18 and shaft separately 14, the device 10 can be reassembled by joining and locking the handle 18 to the shaft 14 for reuse.

Some embodiments may be described using the expression “coupled” and “connected” along with their derivatives. For example, some embodiments may be described using the term “coupled” to indicate that two or more elements are in direct physical contact. The term “coupled,” however, may also mean that two or more elements are not in direct contact with each other, but yet still co-operate or interact with each other. The embodiments are not limited in this context.

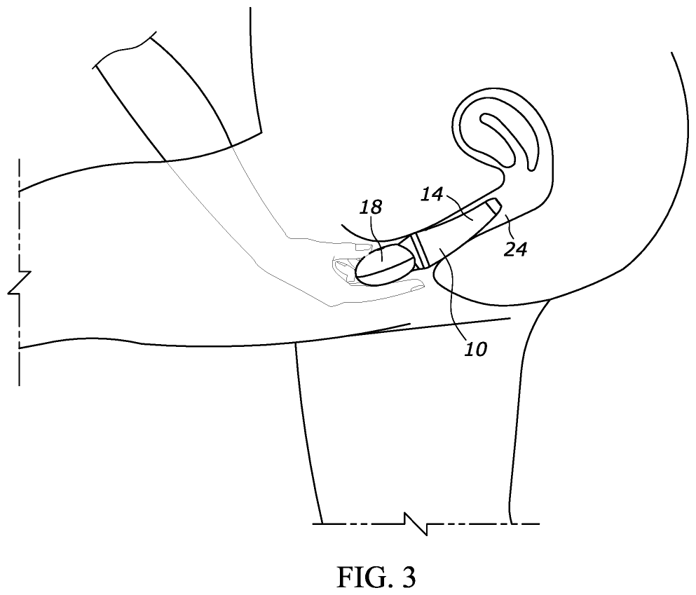

In FIG. 3, the handheld device 10 is shown for use as a vaginal aspirator to remove fluid, such as semen, from the vagina after sex. The shaft 14 is shaped and sized to comfortably enter and gradually widen a vaginal canal of the vagina 24. The handle 18 provides an ergonomic grip and is configured for inducing a suction force at an opening 22 at a distal end 26 of the device 10. When a user compresses the handle 18, inserts the shaft 14 into the vagina 24, and then releases the handle 18 (i.e., permits the handle 18 to return to a non-compressed state), the handle 18 creates a pumping action to induce a soft suction in to pull semen from the vagina 24 and into the device 10. The suction force is soft, so that surrounding tissue is not disturbed or pulled into the shaft 14, but strong enough to pull the semen through the shaft 14 and into an interior volume of the handle 18.

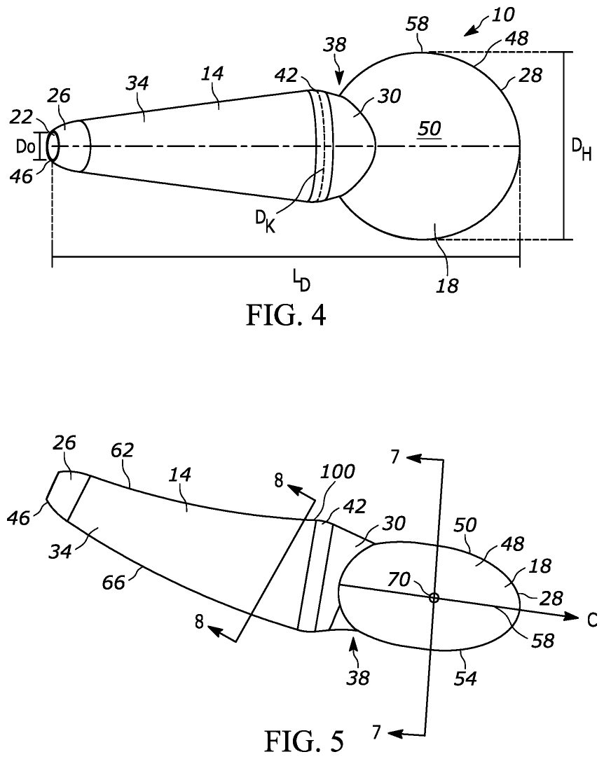

Turning to FIG. 4, the device 10 includes the distal end 26 (or first end 26 of the shaft 14), and an opposite, proximal end 28 defined by the handle 18. The shaft 14 includes a second end 30 opposite the first end 26 of the shaft 14, and a body 34 extending between the first and second ends 26, 30. The body 34 of the shaft 14 is hollow and includes an interior cavity that is in fluid communication with an interior volume of the handle 18. The handle 18 is coupled to the second end 30 of the shaft 14, and engages with the shaft 14 at a mating interface 38. The mating interface 38 provides a seal between the shaft 14 and the handle 18 when the handle 18 and shaft 14 are secured in a locked configuration, as illustrated in FIG. 1, for example.

In FIG. 4, the shaft 14 tapers from a knuckle 42, which as a cross-sectional inner diameter D_{K}, to the opening 22 at the distal end 26, which has a cross-sectional inner diameter D_{O}. The diameter D_{K} is greater than the diameter D_{O} of the opening 22. The device 10 terminates at the distal end 26 with a rounded, blunt tip 46. This blunted tip 46 facilitates penetration of the device 10 into a body cavity without nicking, scraping, or otherwise injuring any surrounding tissue of the body cavity. The gradual taper shape between the knuckle 42 and the tip 46 of the body 34 facilitates comfortable separation of the walls of the body cavity during insertion. The device 10 may be inserted at a desired depth before inducing suction by pumping the handle 18 (i.e., releasing the compressed handle 18). When the device 10 is used as a vaginal aspirator to collect semen in the vagina 24, the semen naturally falls into the opening 22 of the device 10 as the shaft 14 separates the vaginal walls.

FIG. 5 illustrates both the curvature and the tapering of the body 34 of the shaft 14. The body 34 has a first portion 62 that is concave and a second portion 66 that is convex. In this example device 10, the curved shape is designed according to a curvature and angle of the vagina to aid a user in inserting the device 10 into the user's vagina. The orientation of the handle 18 is offset from the curved portions 62, 64 of the body 34 of the shaft 14. This slight offset positioning between the handle 18 and shaft 14, as shown in FIG. 5, positions the handle 18 at an accessible location outside of the body cavity when the device 10 in disposed in a body cavity. So configured, a user can reach the target depth in the vagina while comfortably sitting, legs apart, and without straining or twisting an arm or hand to suitably grip the handle 18. In this example, the device 10 is specifically designed for use with the vagina. However, in other examples, the dimensions, shape, and material may change for use as an anal aspirator.

The handle 18 is ergonomically shaped to comfortably fit in a user's hands. The handle 18 has an adapter 47 (FIG. 2) for coupling the handle 18 to the shaft 14, and a collapsible, ellipsoidal body 48 configured for inducing a suction force in the device 10. As shown in FIGS. 4 and 5, the body 48 of the handle 18 includes first and second grip surfaces 50, 54 that meet at a circumferential edge 58. As shown in FIG. 3, a user may grip the body 48 of the handle 18 with one or more fingers on each gripping surface 50, 54. In particular, and according to the orientation in FIG. 3, a user's thumb may be placed on the first, top gripping surface 50 and the index and middle fingers may be placed on the second, bottom gripping surface 54. Using this grip, the user can compress the body 48 and create a partial void in the device 10 by pressing handle 18 between the thumb and index and middle fingers, and subsequently release the body 48 by permitting the body to return to a decompressed state. In another example, the shaft 14 and handle 18 may be coupled so that the first and second gripping sides or surfaces 50, 54 are oriented 90 degrees about a C axis from what is shown in FIGS. 4 and 5. While the body 48 is a partial ellipsoid, the handle 18 may be arranged to have different geometries, for functional and/or aesthetic purposes, such as, for example, cubic, cylindrical, prismatic, spherical, conical, or other compressible shapes.

The acts of compressing and decompressing (or releasing) of the body 48 creates a pumping function to induce a suction force within the device 10. While the present example utilizes a compressible body 48 of the handle 18 to effectuate a pumping action, other versions can effectuate a pumping action in different ways, such as, for example, sliding pistons, claws, scrolling spirals, diaphragms, or a combination of known methods.

In FIG. 4, a length LD of the device 10 for use as a vaginal aspirator is in a range of approximately 4 inches to approximately 6.75 inches; approximately 5 inches to approximately 6.5 inches; and approximately 5.5 inches to approximately 6.0 inches. In other examples, the length LD of the device 10 may be greater depending on the application of the device 10 (i.e., based on a user's personal body dimensions, body cavity, etc.) and/or a user's preference. The opening 22 of the shaft 14 has an inner diameter D_{O} in a range of approximately 0.05 inches to approximately 0.375 inches; approximately 0.1 inches to approximately 0.35 inches; and approximately 0.2 inches to approximately 0.3 inches. The opening 22 is sufficiently large enough to break a surface tension of a fluid, like semen, to allow the semen to collapse into the opening 22 and cavity of the device 10. Experimentation shows that with an opening 22 too small, the semen will not enter the opening 22 in the shaft 14 as the surface tension of semen resists sliding into the opening 22 of the device 10. The inner diameter D_{K} of the knuckle 42 is in a range of approximately 0.2 inches to approximately 1.5 inches; approximately 0.5 inches to approximately 1.25 inches; and approximately 0.7 inches to approximately 1 inch. An outer diameter DH of the widest portion of the handle 18 is in a range of approximately 1.25 inches to approximately 2.75 inches; approximately 1.5 inches to approximately 2.5 inches; and approximately 1.75 inches to approximately 2.25 inches.

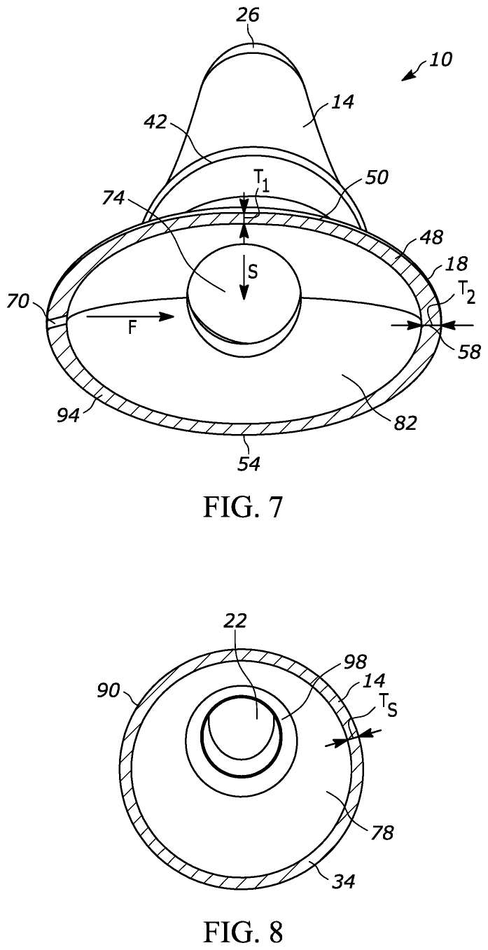

FIG. 5 shows a hole 70 formed in the circumferential edge 58 of the handle 18. The hole 70 creates a passageway into the interior volume of the handle 18, and enables the device 10 to create a suction force when the device 10 is in an air-tight cavity, such as the vagina. Specifically, fluid will not flow through the shaft 14 and into the handle 18 unless there is a passageway for fluid to travel. The hole 70 in the device 10 is located outside of the vagina 24 when the shaft 14 is inserted into the vagina 24 at a desired depth, as shown in FIG. 3. When the user releases the handle 18 (i.e., decompresses the handle body 48), air flows in a direction represented by arrow F through the hole 70 and into the interior volume of the handle 18 (FIG. 7), creating a suction force S in a cavity 74 of the device 10. In other examples, the hole 70 may be placed at a different location on the handle 18 and/or the shaft 14 where the hole 70 will not likely be covered by a user's grip and that is externally located relative to the body cavity when the device 10 is in use.

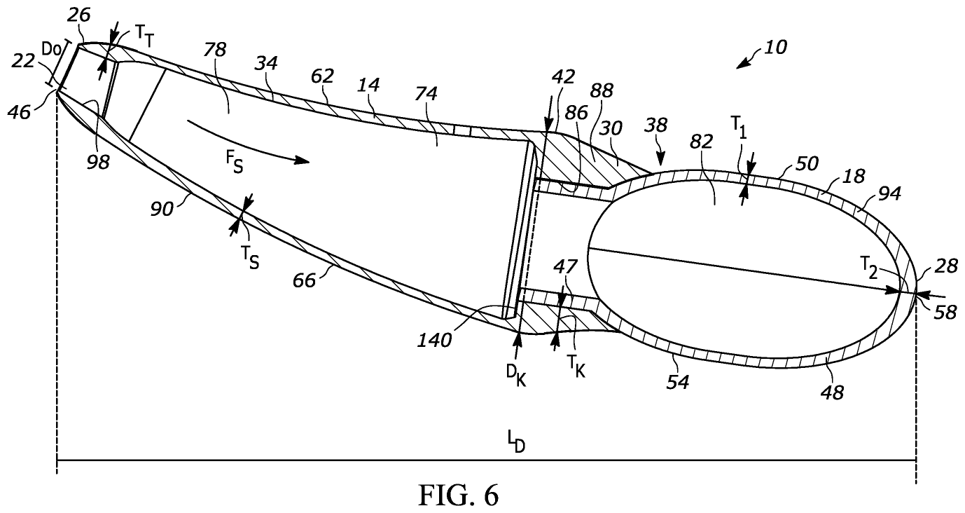

FIGS. 6-8 show various cross-sections of the device 10. When the handle 18 is coupled and locked to the shaft 14, as shown in FIG. 6, an interior cavity 78 of the shaft 14 is in fluid communication with an interior volume 82 of the handle 18 that together form the device cavity 74. In FIG. 6, the adapter 47 of the handle 18 is a male fitting disposed inside a bore 86 of a female fitting 88 of the second end 30 of the shaft 14. The male fitting 47 and female fitting 88 are arranged for locking together the handle 18 and the shaft 14. In other examples, the adapter 47 of the handle 18 and the adapter portion 88, or fitting, of the shaft 14 may be arranged differently.

To achieve different material characteristics in the shaft 14 and the handle 18, the wall thickness and material rigidity of the device 10 can vary. As shown in FIG. 6, the thicknesses of a wall 90 of the shaft 14 and a wall 94 of the handle 18 vary at different locations of the device 10. For example, each of the thicknesses T₁, T₂ of a wall 94 of the handle 18 is greater than a thickness T_{S} of a wall 90 of the shaft 14 between the tip 46 and the knuckle 42. Additionally, a hardness of the material, which in this case is medical grade silicone, may be different between the shaft 14 and the handle 18. Accordingly, while the average thickness of the handle 18 is greater than an average thickness of the shaft 14 (between the tip 46 and the knuckle 42), the body 48 of the handle is compressible and pliable, whereas the shaft 14 of the device 10 is rigid and resists compression. The handle 18 may be formed of a medical grade silicone, such as 80A, so that the body 48 is sufficiently pliable to permit compression, yet resists permanent, or at least readily detectable, deformation. The shaft 14 may be formed of a different grade silicone, such as 100A, so that the shaft 14 resists compressive forces of a user's body and maintains its shape when inserted in the body cavity.

In FIGS. 6 and 8, the blunt tip 46 of the device 10 defines an interior collar 98 that provides additional rigidity to the distal end 26 of the shaft 14. The collar 98 provides an added wall thickness TT to the first end 26 of the shaft body 34 to facilitate insertion of the device 10 through a muscular body cavity. At an example location 100 between the knuckle 42 and the collar 98, the body 34 has a thickness T_{S} that is less than a thickness TT of the wall 94 at the tip 46. A wall thickness TK of the knuckle 42 is greater than both the thickness TT of the collar 98 and the thickness T_{S} of the other portions of the body 34. The wall thickness TK of the knuckle 42 and portions adjacent the knuckle 42 provide added rigidity to the mating interface 38 of the device 10. The thickest, and most rigid portion of the device 10, is at the mating interface 38, where the adapter 47 of the handle 18 overlaps with the fitting 88 of the shaft 14. The mating interface 38 of the device 10 is sufficiently rigid to provide a secure lock when the handle 18 and shaft 14 are coupled.

Turning to FIG. 7, the wall thickness of the handle 18 varies. For example, the wall 94 closest to the gripping surfaces 50, 54 of the handle 18 has a thickness T₁, which gradually increases towards the circumferential edge 58 of the body 48 (FIG. 6). In other words, the thinnest, most pliable portion of the handle 18 is centrally located relative to the first and second surfaces 50, 54 to facilitate manual pumping. A thickness T₂ of the circumferential edge 58 of the handle 18 provides rigidity and resists deformation when the handle 18 is pumped. Further, the thickness T₂ at the circumferential edge 58 also discourages improper use of the handle 18. In the illustrated example, both the shaft 14 and the handle 18 have wall thicknesses that vary, or, in other words, are not uniform throughout. However, in other examples, the wall thickness of the shaft 14, the handle 18, or both shaft 14 and handle 18 may be uniform. In an example of an integrated device, where the shaft 14 and handle 18 are not separable and are made of the same medical grade silicone, the wall thickness may be similar to the thicknesses in FIGS. 6-8, have a different variability, or may be uniform using a different grade or material.

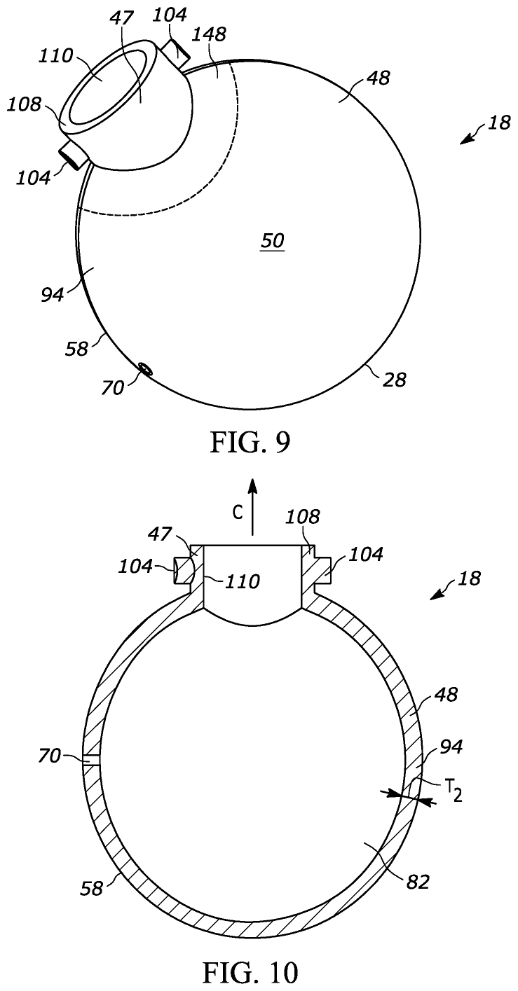

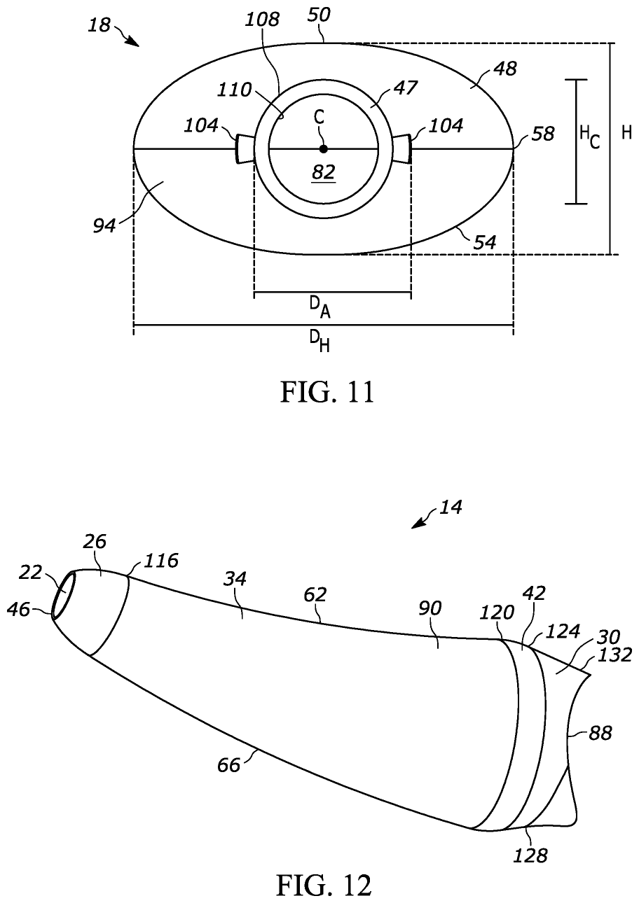

In FIGS. 9-11, the handle 18 is illustrated separately from the shaft 14. The handle 18 has a disc-shaped or ellipsoidal body 48 that defines the interior volume 82. The interior volume 82 is sized to induce a soft suction force without creating a strong suction force that could harm the body cavity. As shown in FIG. 10, the thickness T₂ of the edge 58 (excluding the adapter 47) is uniform, whereas the cross-section of the body 48 in FIG. 7 shows the wall 94 having a varying thickness. A height H of the handle 18 in a non-compressed state, as shown in FIG. 11, is in a range of approximately 0.5 inches to approximately 2.25 inches; approximately 0.75 inches to approximately 2 inches; and approximately 1 inch to approximately 1.5 inches. When the handle 18 is compressed, a height H_{C} of the handle 18 is in a range of approximately 0.1 inches to approximately 1.5 inches; approximately 0.25 inches to approximately 1.25 inches; and approximately 0.5 inches to approximately 1 inch.

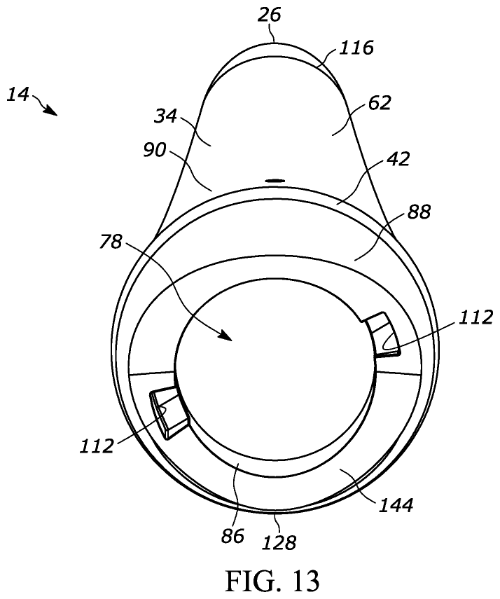

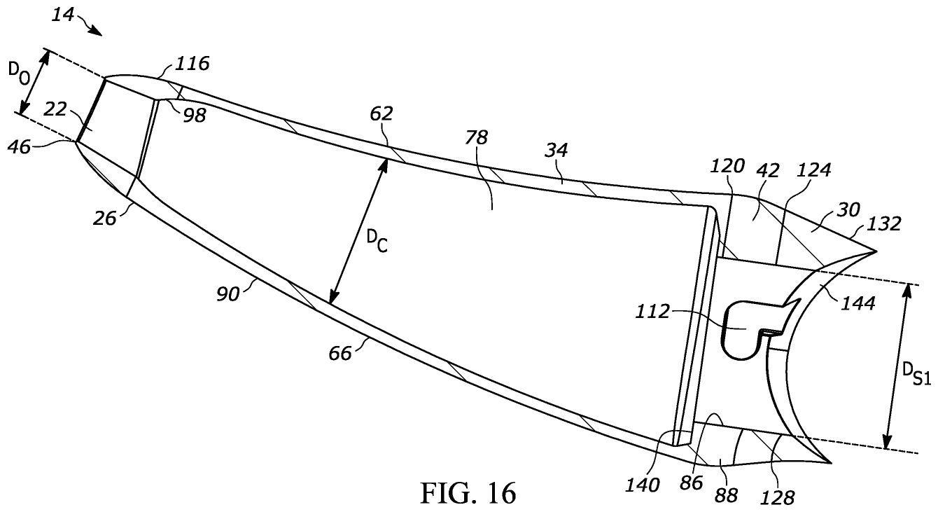

The adapter 47 in FIGS. 9-11 is a male luer-lock fitting with two pegs 104 protruding from a cylindrical portion 108. The cylindrical portion 108 has an outer diameter D_{A} that is sized to fit within the bore 86 of the shaft 14 for coupling with the female luer-lock fitting 88 of the shaft 14, and the pegs 104 are sized to slide and rotate within L-shaped tracks 112 (FIGS. 12 and 16) formed in the female-fitting 88 of the shaft 14. The cylindrical portion 108 defines an interior bore 110 of the adapter 47 that provides fluid communication between the cavity 78 of the shaft 14 with the interior volume 72 of the handle 18. The outer diameter D_{A} of the adapter 47 is in a range of approximately 0.1 inches to approximately 1 inch; and approximately 0.25 inches to approximately 0.75 inches.

In FIGS. 12-16, the shaft 14 is illustrated separately from the handle 18. While formed as an integrated piece, the shaft 14 includes an insertion portion (from the tip 46 to adjacent to the knuckle 42), the adapter portion 88 that defines the second end 30, and the knuckle 42 connecting the insertion portion with the adapter portion 88. The second end 30 of the shaft 14 terminates in a curved or convex opening so that a length of the shaft 14, measured from the distal point of the first end 26 to the proximal point of the second end 30, varies between L_{S1} and L_{S2}. The shaft 14 has a length L_{S1} defined between the tip 46 and an outermost edge of the second end 30, and a length L_{S2} defined between the tip 46 and an innermost edge of the second end 30. The length L_{S1} is in a range of approximately 3 inches to approximately 4.5 inches; approximately 3.25 inches to approximately 4.25 inches; and approximately 3.5 inches to approximately 4 inches. The length L_{S2} is in a range of approximately 2.5 inches to approximately 4 inches; approximately 2.75 inches to approximately 3.75 inches; and approximately 3 inches to approximately 3.5 inches.

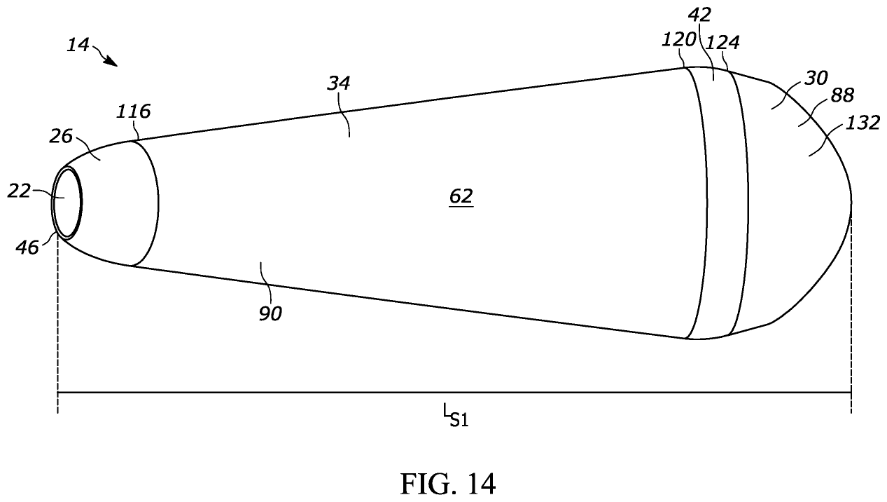

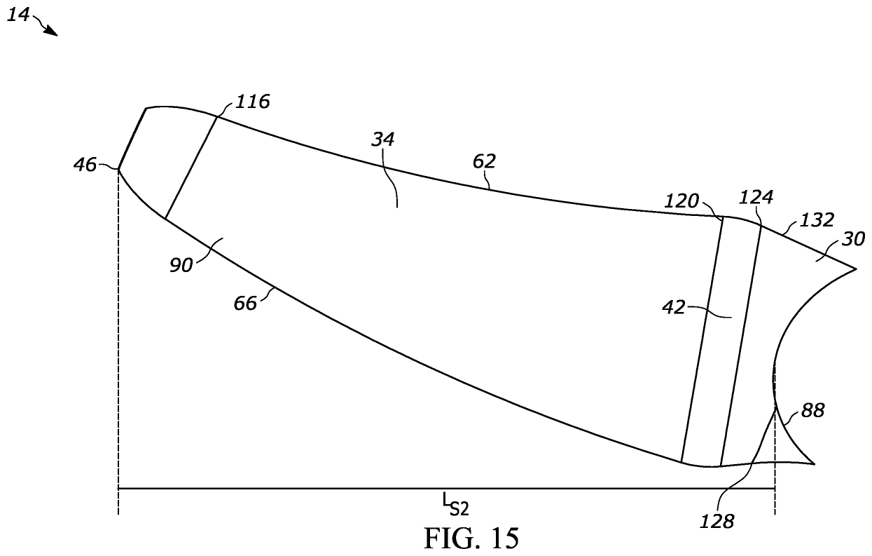

The shaft 14 generally has the first (top) side curvature 62, and second (bottom) side curvature 64 (according to the orientation in FIGS. 15 and 16), and the overall shape of the body 34 is defined in part by three points of inflection on the first portion 62 of the shaft 14. In other words, the curvature of the outer surface of the shaft 14 changes at three different locations. A first point of inflection 116 is at the first end 26 of the shaft 14. The first point of inflection 116 transitions the concave curvature 62 of the body 34 to a convex portion to form the rounded, blunted tip 46. The first point of inflection 116 provides a smooth outer surface of the shaft 14 to facilitate insertion of the device 10 without nicking, scratching, or scraping any body tissue. A second point of inflection 120 provides a smooth transition between the concave curvature 62 of the body 34 to a convex curvature of the knuckle 42. A third point of inflection 124 transitions the convex curvature of the knuckle 42 to an angled, or slightly concave portion of the second end 30 of the shaft 14. At the second end 30, the body 34 may be slightly concave at a lower portion 128, or inflection point, to transition the adapter of the shaft 14 to the second side of the handle 18. Whereas, an upper portion 132 of the second end 30 of the shaft 14 may not have any curvature. As shown in FIG. 14, the second end 30 of the shaft 14 tapers from the knuckle 42 to mating interface 38 and from the knuckle 42 to the tip 46. Specifically, a midpoint of the knuckle 42 (i.e., the midpoint between the second and third points of inflections 120, 124) defines the outer diameter of the knuckle D_{K} and the location in which the body 34 begins to taper toward opposing first and second ends 26, 30 of the shaft 14.

Turning to FIG. 16, the various internal features of the shaft 14 are illustrated. Specifically, the wall thicknesses and inner diameters vary across the length of the shaft 14. The opening 22 has the inner diameter D_{O}, which is defined in part by the collar 98, and a diameter D_{C} of the interior cavity 78 increases from the opening 22 to a location adjacent to the knuckle 42. Where the wall 90 forms a shoulder 140, the cavity 78 has a substantially constant inner diameter D_{S1}, which is also the diameter of the bore 86 of the second end 30. At the shoulder 140, the thickness of the wall 90 abruptly increases and then tapers toward the second end 30. The L-shaped tracks 112 are formed in the bore 86 of the shaft 14, and are and shaped to receive the pegs 104 of the handle 18 and tightly secure the handle 18 to the shaft 14 when the handle 18 is twisted to lock the handle 18 and shaft 14 together. The diameter D_{S1} of the bore 86 is slightly greater than the outer diameter D_{A} of the handle fitting 47 so that the male and female fittings 47, 88 of the device 100 lock by friction.

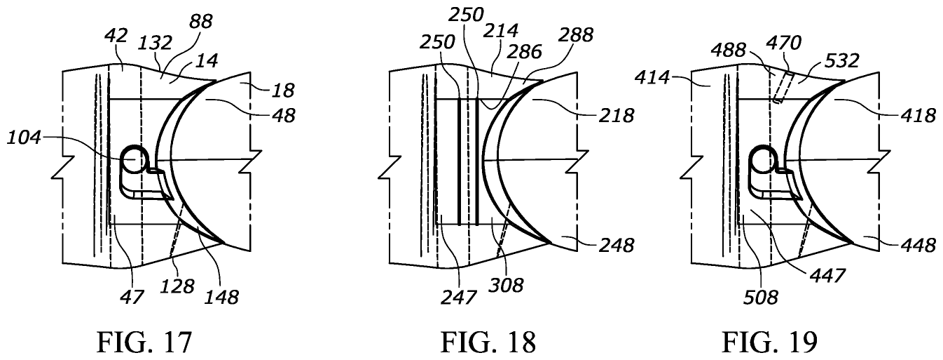

The mating interface 38 includes a mating surface 144 at the second end 30 of the shaft 14 (FIGS. 13 and 16) and a mating surface 148 (FIG. 9) of the handle 18. When the handle 18 is coupled and locked to the shaft 14, as shown in FIG. 17, the mating surfaces 144, 148 abut one another to provide a snug fit. In FIG. 17, the shaft 14 is transparent to illustrate the fit between the mating surfaces 144, 148. As shown, the mating surface 144 of the shaft 14 is concave and beveled to sealingly engage with the convex mating surface 148 of the ellipsoid body 48 of the handle 18. The fitting 88 of the second end 30 is asymmetrical about an axis through the bore 86, where the lower portion 128 is slightly concave, and the upper portion 132 is angled. When coupled, a distal end of the adapter of 47 the handle 18 is coplanar with the interior shoulder 140 of the shaft 14 (FIG. 6).

While the present example utilizes a rotatable locking mechanism between the handle 18 and the shaft 14 to effectuate a locking engagement, other versions can effectuate a locking engagement in different ways, such as, for example, a different male-female locking mechanism and/or arrangement, a ball-and-socket, snap-fit, push-fit, interference fit, threading, or a combination of known methods. In FIG. 18, for example, a shaft 214 is coupled to a handle 218 by a snap-fit engagement. In this example, an adapter 247 of the handle 218 includes seals 250, such as O-rings, or annular ridges, that provide either a sealing engagement with a bore 286 of the shaft 214 or that snap into corresponding grooves formed in the bore 286 of the shaft 214. Other examples may include other coupling mechanisms of varying materials.

FIG. 19 illustrates a third example mating interface 438 between a shaft 414 and a handle 418 where a vent hole 470 that facilitates suction of the device is disposed in both a cylindrical portion 508 of an adapter 447 of the handle 418 and a top portion 532 of a fitting 488 of the shaft 414.

The device 10 is made of a medical grade silicone (100A and/or 80A) that is anti-bacterial and nonporous. The shaft 14 and handle 18 are both made of silicone, but made from a different grade to control rigidity of each component. For example, a combination of the thickness of the shaft wall 90 and the grade of the silicone of the body 34 provides the shaft 14 with enough rigidity and strength to withstand contracting or tightening of a body cavity wall (e.g., the vagina or sphincter). The device may include a surface treatment that provides a smooth, satin finish for to facilitate insertion of the device 10 and to help the locking mechanism lock and unlock easily and securely.

The device 10 is made of medical grade silicone, but can be made in other non-porous materials that are safe for insertion into the body and easy to clean. The device 10 may be manufactured from any suitable material, but is preferably formed of silicone using liquid silicone injection methods. However, the device 10 may also be made from an extrudable material including, but not limited to, extrudable polymers and metals. Exemplary extrudable plastics include, but are not limited to, polyvinylchlorides, polyethylenes, polypropylenes, acetals, acrylics, nylons (polyamides), polystyrene, acrylonitrile butadiene styrenes, and polycarbonates.

Further, the device 10 may be formed using an additive manufacturing technique or process that builds three-dimensional objects by adding successive layers of material on a material. The additive manufacturing technique may be performed by any suitable machine or combination of machines. The additive manufacturing technique may typically involve or use a computer, three-dimensional modeling software (e.g., Computer Aided Design, or CAD, software), machine equipment, and layering material. Once a CAD model is produced, the machine equipment may read in data from the CAD file and layer or add successive layers of liquid, powder, sheet material (for example) in a layer-upon-layer fashion to fabricate a three-dimensional object. The additive manufacturing technique may include any of several techniques or processes, such as, for example, a stereolithography (“SLA”) process, a fused deposition modeling (“FDM”) process, multi-jet modeling (“MJM”) process, a selective laser sintering (“SLS”) process, an electronic beam additive manufacturing process, and an arc welding additive manufacturing process. In some embodiments, the additive manufacturing process may include a directed energy laser deposition process. Such a directed energy laser deposition process may be performed by a multi-axis computer-numerically-controlled (“CNC”) lathe with directed energy laser deposition capabilities. Other additive manufacturing techniques are also contemplated.

Turning back to FIG. 3, a method of using the device 10 as a vaginal aspirator to remove semen after sex will be described. Initially, a user may while sitting or standing, separate their legs approximately 45 degrees apart or in another manner that is comfortable for the user. The user may grip the handle 18 using two or more fingers and compress the body 48 of the handle 18 before inserting the device 10 into the vagina 24. While the handle 18 is in the compressed configuration, the user can align the tip 46 of the device 10 with a vaginal opening and insert the shaft 14 into the vagina 24. When the device 10 is inserted (i.e., when the knuckle 42 is adjacent or near the vaginal opening), the user can release the compression against the handle 18. The handle 18 will inflate, creating a soft suction to pull semen into the opening 22 in the shaft 14 and collect the semen in the cavity 74 (e.g., the interior volume 82 of the handle 18) of the device 10, at which point the user can remove the shaft 14 from the vagina 24.

The device 10 and its different iterations disclosed and described herein provide a number of benefits over the prior art. The device 10 is shaped to comfortably reach the depths of a body cavity, and provide a gentle suction for simple, post-sex clean-up. The device 10 is simply constructed with two components that easily lock together, and each part has smooth surfaces and rounded edges. Further, the device 10 may be adapted for use for either a vaginal or anal aspirator, and may be used in personal or medical applications.

The device 10 is easy to use and pump with one hand. As shown in FIG. 3, the vagina 24 is angled relative to a person's spine, and the orientation of the handle 18 and curvature of the shaft 14 are positioned to reduce any strain and discomfort on the user. For example, as a user is sitting and looking down toward their pelvic region, the user can easily hold and manipulate the handle 18 and insert the curved shaft 14, which is comfortably guided by the curvature and angle of the vagina 24. In other words, the configuration of the curvature of the shaft 14 matches the orientation of the vagina while placing the handle 18 at an accessible and comfortable position outside of the vagina 24.

The device 10 is also made of a non-porous, medical grade material that is safe for use within the body and easily disassembled for cleaning. The cavity 78 of the shaft 14 is sized to receive a cleaning brush and/or an average sized finger to facilitate cleaning. Accordingly, the device 10 can be cleaned and reused multiple times for a sustainable personal hygiene solution.

Finally, although a fluid collection device has been described herein in accordance with the teachings of the present disclosure, the scope of coverage of this patent is not limited thereto. On the contrary, while the disclosed device has been shown and described in connection with various examples, it is apparent that certain changes and modifications, in addition to those mentioned above, may be made. This patent application covers all examples of the teachings of the disclosure that fairly fall within the scope of permissible equivalents. Accordingly, it is the intention to protect all variations and modifications that may occur to one of ordinary skill in the art.

What is claimed:

1. A body cavity fluid removal device, the device comprising:
a shaft including a first end, a second end opposite the first end, and an opening at the first end;
a handle including a compressible body defining an interior volume, the handle being removably coupleable to the second end of the shaft;
wherein the handle is configured for inducing a suction force at the opening of the shaft.

2. The device of claim 1, wherein when the handle is coupled to the shaft, the interior volume is in fluid communication with the opening of the shaft.

3. The device of claim 1, wherein the shaft and pump couple by a rotatable locking mechanism.

4. The device of claim 1, wherein when the pump and shaft are securely coupled, an adapter of the handle is disposed in a bore defined by the second end of the shaft.

5. The device of claim 1, wherein the shaft is curved.

6. The device of claim 1, wherein the shaft includes a wall having a variable thickness between the first end and the second end.

7. The device of claim 1, further comprising a hole disposed through a wall of the handle and in fluid communication with a cavity of the shaft.

8. The device of claim 1, further comprising a first hole disposed through a wall of the pump and a second hole disposed through a wall of the shaft, such that when the shaft and the pump are securely coupled, the first and second holes align to form a continuous vent in fluid communication with an interior cavity of the shaft.

9. The device of claim 1, wherein the second end defines a mating surface and the pump defines a corresponding mating surface, the mating surfaces sealing engage when the shaft and pump are coupled.

10. An aspirator for removing fluid from a body cavity, the device comprising:
a shaft including a first end, a second end, and a body having a curved surface extending between the first and second ends, the body defining a cavity; and
a handle coupleable to the second end of the shaft, the second end of the shaft including an interior volume in fluid communication with the cavity of the shaft when the handle and shaft are coupled.

11. The device of claim 10, wherein the curved surface includes a point of inflection at the first end of the shaft.

12. The device of claim 10, wherein the shaft is a medical grade silicone.

13. The device of claim 10, wherein when the handle is compressed and released, a suction force is created in the cavity of the shaft.

14. The device of claim 10, wherein the body is tapered and configured for insertion into a vaginal canal.

15. The device of claim 10, wherein the first end defines an opening having a diameter in a range of approximately 0.1 inches to 0.3 inches.

16. The device of claim 10, further comprising a hole disposed through the wall of the handle, the hole in fluid communication with the interior volume.

17. The device of claim 10, further comprising a coupling mechanism arranged for removably coupling the handle to the shaft.

18. A method of removing fluid from a body cavity, the method comprising:
inserting a shaft of a handheld device into a body cavity, the shaft including a first distal end defining an opening and a second end coupled to a handle, the shaft including a bore and a curved surface; and
activating a suction force through the shaft.

19. The method of claim 18, wherein activating a suction force includes compressing a flexible body of the handle externally disposed relative to the body cavity, the handle including a body defining an interior volume in fluid communication with the bore of the shaft.

20. The method of claim 19, further comprising releasing the handle, causing fluid disposed in the body cavity to enter the shaft through the opening and be received by the interior volume of the handle.
